(19) United States
(12) Patent Application Publication
(10) Pub. No.: US 2002/0193618 A1
(43) Pub. Date: Dec. 19, 2002

(54) Process for the preparation of saturated organic compounds

(76) Inventors: Daniel Ostgard, Kleinostheim (DE); Monika Berweiler, Maintal (DE); Stefan Roder, Sinntal (DE)

Correspondence Address: SMITH, GAMBRELL & RUSSELL, LLP, 1850 M Street, N.W., Suite 800, Washington, DC, 20036, US

(21) Appl. No.: 10/034,989

(22) Filed: Jan. 3, 2002

(30) Foreign Application Priority Data
Jan. 16, 2001 (DE) 101 01 646.8

(51) Int. Cl. C11C 3/12; C07C 2/56; C07C 27/04

(52) U.S. Cl. 554/141; 568/885; 585/711

(57) ABSTRACT

Process for the preparation of completely or partly saturated organic compounds by catalytic hydrogenation of unsaturated organic compounds with hydrogen or hydrogen-containing gas mixtures in the presence of a shaped Raney catalyst as the hydrogenation catalyst, wherein the Raney catalyst is in the form of hollow bodies. Nickel, cobalt, copper, iron, platinum, palladium or ruthenium are preferably used as catalytically active constituents.

The invention relates to a process for the preparation of completely or partly saturated organic compounds by catalytic hydrogenation of unsaturated organic compounds.

The preparation of saturated organic compounds is of decidedly high importance in industry. Two important examples which may be mentioned are the hydrogenation of fats or the preparation of alicyclic compounds by hydrogenation of unsaturated fats or aromatic starting compounds.

Raney catalysts are often preferably employed in the preparation of saturated organic compounds by hydrogenation of unsaturated organic compounds because of their good catalytic properties. Raney catalysts, which are also called activated metal catalysts, comprise an alloy of at least one catalytically active metal and at least one metal which can be leached out with alkalis. Aluminium is predominantly employed for the alloy component which is soluble in alkalis, but other metals, such as, for example, zinc and silicon, can also be used. The component which can be leached out is dissolved out by addition of alkalis to the alloy, as a result of which the catalyst is activated.

Numerous processes for the preparation of saturated organic compounds by catalytic hydrogenation with the aid of Raney catalysts are known. Various Raney catalysts, or more precisely catalysts with various active metals or metal combinations, are employed here, depending on the process.

For example, Wang et al. (Wang, Chengxue; Hua, Xuan; Zhang, Ying; Shiyou Huagong (1992), 21 (6), 359-63) describe the hydrogenation of butinediol to butanediol with the aid of a nickel Raney catalyst.

The document EP 0 724 908 describes the hydrogenation of aromatic compounds with the aid of pulverulent Raney catalysts which comprise ruthenium as the catalytically active component. The hydrogenation is used on unsubstituted hydrocarbons, such as, for example, biphenyl, and also a large number of variously substituted aromatic compounds, such as phenol, diphenyl ether or toluene. Aromatic compounds which can be hydrogenated to the corresponding saturated substances are both carbocyclic compounds and heteroaromatics, such as, for example, pyridines.

Raney powder catalysts have the disadvantage that they can be employed only in the batch process or at best in the semi-continuous process if sufficient conversion rates are to be achieved under moderate reaction conditions. Furthermore, the catalysts must be separated off from the reaction media in an expensive manner after the catalytic reaction. For these reasons also, it is preferable to carry out the preparation of completely or partly saturated organic compounds by hydrogenation of unsaturated organic compounds with the aid of shaped Raney catalysts and where possible in a continuous process. Fixed bed catalysts which, in addition to a good catalytic activity, must also have a sufficiently high strength for the continuous operation are needed for this purpose.

The document U.S. Pat. No. 6,018,048 describes the hydrogenation of aromatic compounds in a continuous process in which a ruthenium Raney catalyst is employed. This catalyst is present in the form of granules in a fixed bed in the continuous procedure. The catalytically active regions of granulated Raney catalysts usually lie only in a more or less thick layer on the surface of the granules. A disadvantage of the process described in U.S. Pat. No. 6,018,048 is therefore that relatively high portions of the catalyst cannot have a catalytic action. The catalyst activity thus falls, and larger amounts of catalyst are required. Another disadvantage of the known process is that the catalyst granules used have a relatively high bulk density of above approx. 1.3 g/ml. As a result, for example, particular requirements are imposed on the reactor in respect of stability.

The document JP 09132536 A2 describes a process for the hydrogenation of compounds with aromatic or aliphatic multiple bonds in a continuous process. The nickel Raney catalyst used in this process can be employed in two successive steps, first in the fixed bed in the form of catalyst lumps and then, after grinding and renewed activation, as a powder catalyst. Approximately the total potential of catalytically active metal is indeed used as the catalyst in successive steps in this process. However, the intermediate step of grinding and subsequent activation is very expensive. On the other hand, if only the preferred continuous process is considered, the low activity of the catalyst lumps is a disadvantage because of the high content of non-activated metal alloy and the high bulk density.

The document DE 199 33 450.1 describes metal catalysts which are in the form of hollow bodies, preferably in the form of hollow spheres. These catalysts have a low bulk density of 0.3 to 1.3 g/ml. In addition to the catalysts, their use in hydrogenation reactions is furthermore claimed. The examples describe an activity test for the hydrogenation of nitrobenzene to aniline, in which the hydrogen consumption and therefore the activity of the catalyst per gram of catalyst is significantly higher if catalysts in the form of hollow spheres are used than if a comparison catalyst is used. However, the use of the catalysts described for the preparation of completely or partly saturated organic compounds by hydrogenation of unsaturated organic compounds is not mentioned.

The object of the invention is to develop a process for the preparation of completely or partly saturated organic compounds, in particular completely saturated organic compounds, by catalytic hydrogenation of unsaturated organic compounds, in which the hydrogenation is carried out with a shaped hydrogenation catalyst of the Raney type which, with an adequate strength and a substantially lower bulk density than comparable catalysts, has the same or a better hydrogenating activity than the catalysts used hitherto. Another object of the invention is to achieve the same or better conversion rates of the starting materials using less catalyst material compared with known processes.

According to the invention, it has been found that the preparation of saturated organic compounds by hydrogenation of unsaturated organic compounds is possible with significantly higher conversion rates with the aid of the Raney catalysts in the form of hollow bodies described in the document DE 199 33 450.1 than with comparable catalysts. This observation is surprising in that it cannot necessarily be assumed that the Raney catalysts in the form of hollow bodies have the required activities and selectivities in the specific case of hydrogenation of unsaturated organic compounds.

The invention provides a process for the preparation of completely or partly saturated organic compounds by catalytic hydrogenation of unsaturated organic compounds with hydrogen or hydrogen-containing gas mixtures in the presence of a shaped Raney catalyst as the hydrogenation catalyst, which is characterized in that the Raney catalyst is in the form of hollow bodies.

According to this invention, organic compounds with unsaturated regions are understood as meaning those compounds which contain at least one C—C multiple bond, in particular a C—C double bond, a C—C triple bond or aromatic C—C bonds.

According to the invention, completely saturated organic compounds are understood as meaning those organic compounds which contain no C—C multiple bond, in particular no C—C double bond, no C—C triple bond and no aromatic C—C bonds. Partly saturated organic compounds are understood as meaning those organic compounds which, compared with the starting compound of the process according to the invention, contain fewer C—C multiple bonds, in particular fewer C—C double bonds, fewer C—C triple bonds and fewer aromatic C—C bonds.

The process according to the invention has the advantage that completely or partly saturated organic compounds can be prepared with equally good or higher yields using significantly smaller amounts of catalyst than has hitherto been possible according to the prior art.

The advantage on which the invention is based is achieved by the use of Raney catalysts in the form of hollow bodies.

The preparation of the catalysts used in the process according to the invention can be carried out according to the method described in DE 199 33 450.1. According to this method, a mixture of an alloy powder of a catalytically active metal with a metal which can be leached out, preferably aluminium, an organic binder and optionally an inorganic binder, water and promoters is applied to spheres which are preferably made of a material which can be removed by means of heat. Polystyrene foam spheres can particularly preferably be used. The mixture comprising the metal alloy can preferably be applied to the polymer spheres in a fluidized bed. 0-10 wt. % polyvinyl alcohol and/or 0-3 wt. % glycerol can preferably be employed as the organic binder. The coated polymer foam spheres are then calcined above 300° C., preferably in a range from 450 to 1300° C., in order to remove the polymer foam by means of heat and to sinter the metal. The hollow spheres acquire a stable form as a result. After the calcining, the catalysts in the form of hollow spheres are activated by treatment with basic solutions, preferably alkali metal or alkaline earth metal hydroxides in water, more preferably aqueous sodium hydroxide solution. They can then be washed with water until the pH is less than 9. The catalysts obtained in this way have bulk densities of between 0.3 and 1.3 kg/l.

For the process according to the invention, it is preferable for the Raney catalysts in the form of hollow bodies to comprise nickel, cobalt, copper, iron, platinum, palladium, ruthenium or mixtures of these metals as catalytically active constituents.

Those Raney catalysts which have been activated by leaching out aluminium, silicon and/or zinc, in particular aluminium, by means of alkalis are preferably used in the preparation according to the invention of saturated organic compounds. The activation can preferably be carried out with aqueous solutions of sodium hydroxide. In this case, the weight ratio of water to alkali metal hydroxide is in general approximately 10:1 to about 30:1, preferably approximately 15:1 to 25:1. The molar ratio of alkali metal hydroxide to aluminium is as a rule 1:1 to approximately 6:1, preferably approximately 1.5:1 to approximately 3:1.

According to the invention, the process is carried out with catalysts in the form of hollow bodies. It is preferable for the Raney catalysts to be in the form of hollow spheres. Hollow spheres are usually easy to produce and have a high breaking strength.

An important advantage of the process according to the invention is that the Raney catalysts used have a lower bulk density than the Raney catalysts known from the prior art for the hydrogenation of unsaturated organic compounds. It is advantageous that the bulk density of the Raney catalysts used is in the range from 0.3 g/ml to 1.3 g/ml.

If catalyst shaped articles which are too large are used, the educt to be hydrogenated possibly cannot come into contact with the catalyst to a sufficient extent. A particle size of the catalysts which is too small means that a very high pressure loss, possibly too high, occurs in the continuous procedure. It is therefore preferable for the catalyst shaped articles used to have a diameter in the range from 0.05 to 20 mm.

So that the catalysts employed in the process according to the invention have on the one hand an adequate strength and on the other hand a low bulk density, it is preferable for the catalyst shaped articles used to have a shell thickness in the range from 0.05 to 7 mm, preferably 0.1 mm to 5 mm. A lower shell thickness can lead to an inadequate breaking strength of the catalyst hollow bodies.

The catalyst shells can be impermeable or can have a porosity of 0% to 80% and higher.

Catalysts in the form of hollow bodies which comprise one or more layers can be used in the process according to the invention. If the catalyst hollow bodies have several layers, the shaped articles are coated in several steps during the preparation and dried between the individual coating steps. The drying is preferably carried out in a fluidized bed at temperatures of 60 to 150° C.

It is possible for the activated catalyst shaped articles used in the process to comprise an inorganic binder. The binder enables the catalyst hollow bodies to have a higher strength, which is necessary due to their hollow form. Preferably, powders of the metals which are also contained in the catalyst alloy as catalytically active constituents are added as binders during the preparation of the catalyst hollow bodies. However, it is also possible to add other binders, in particular other metals, as binders.

It is often also advantageous for the activated catalyst shaped articles used in the process to comprise no binder. If cobalt catalysts are employed according to the invention for the preparation of completely or partly unsaturated organic compounds, these are preferably employed without a binder. Cobalt catalysts in the form of hollow bodies can also have an adequate strength without an added binder.

The catalyst alloy of the catalysts used according to the invention is preferably composed to the extent of 20-80 wt. % of one or more catalytically active metals and to the extent of 20-80 wt. % of one or more metals which can be leached out with alkalis, preferably aluminium. A rapidly or a slowly cooled alloy can be used as the catalyst alloy. Rapid cooling is understood as meaning, for example, cooling at a rate of 10 to 10⁵ K/s. Cooling media can be various gases or liquids, such as, for example, water. Slow cooling is understood as meaning methods with lower cooling rates.

Raney catalysts in the form of hollow bodies which are doped with other metals can be used in the process according to the invention. The doping metals are often also called promoters. The doping of Raney catalysts is described, for example, in the documents U.S. Pat. No. 4,153,578, DE 21 01 856, DE 21 00 373 or DE 20 53 799. The Raney catalyst in the form of hollow bodies used can preferably be doped with one or more elements from groups 3B to 7B, 8 and 1B of the periodic table, in particular chromium, manganese, iron, vanadium, tantalum, titanium, tungsten, molybdenum, rhenium and/or metals of the platinum group. It is also possible, but less preferred, for the Raney catalyst in the form of hollow bodies used to be doped with one or more elements from groups 1A, 2A, 2B and/or 3A of the periodic table and/or germanium, tin, lead, antimony or bismuth. The content of promoters in the catalyst can preferably be 0-20 wt. %. The promoters can already be contained in the catalyst as an alloy constituent, or can be added only at a later point in time, in particular after the activation.

The Raney catalysts in the form of hollow bodies are employed in the activated form during the process according to the invention. The metal which can be leached out and is present in the non-activated catalyst shaped articles can have been leached out with alkalis completely or only partly in the activated state.

The process according to the invention can be carried out with hydrogen as the hydrogenating gas or with gas mixtures which comprise hydrogen, for example a mixture of hydrogen and nitrogen and/or carbon dioxide. In order to avoid possible poisoning of the catalyst, it is preferable to carry out the process according to the invention with a gas or gas mixture comprising at least 95%, preferably at least 99% hydrogen.

The process allows the preparation of more or less pure individual substances and also the preparation of mixtures of variously saturated compounds.

It is preferable for the hydrogenation to be carried out in a fixed bed or suspension reactor in continuous operation. However, the invention also provides for carrying out the hydrogenation in the batch process. In the continuous procedure, the reactor can be operated in the liquid phase process or in the trickle bed process, the trickle bed process being preferred. Reactors and precise methods of carrying out the reaction are known.

The process according to the invention can be carried out with unsaturated aliphatic, cycloaliphatic and aromatic starting compounds. It is possible for the unsaturated organic compounds to carry one or more substituents. These substituents can be, independently of one another, for example, alkyl, cycloalkyl, aryl, heteroaryl, alkenyl, alkinyl F, Cl, Br, I, NO₂, NH₂, NHR, NR₂, OH, HS, S═C, R—CO—O, R—SO, R—SO_{2,} CN, O═CR, HOOC, H₂NOC, ROOC or RO radicals, where R =alkyl, cycloalkyl, aryl, alkenyl, alkinyl, amino, alkylamino. The radical R can optionally carry further substituents, such as, for example, alkyl, cycloalkyl, aryl, alkenyl, alkinyl F, Cl, Br, I, NO₂, NH₂, NHalkyl, NHaryl, Nalkyl₂, Naryl₂, OH, HS, S═C, alkyl-CO—O, aryl-CO—O, alkyl-SO, aryl-SO, alkyl-SO₂, aryl-SO₂, CN, O═Calkyl, O═Caryl, HOOC, H₂NOC, alkylOOC, arylOOC, alkylO, cycloalkylo or arylO radicals.

If aromatic starting compounds are used, these can be mono- or polynuclear, carbocyclic or heterocyclic and five- or six-membered. Examples of suitable substance classes as the starting compound are benzene and its derivatives, substituted and unsubstituted pyridines, substituted and unsubstituted pyridazines, substituted and unsubstituted pyrimidines, substituted and unsubstituted pyrazines, substituted and unsubstituted triazines, substituted and unsubstituted naphthalenes, substituted and unsubstituted quinolines, substituted and unsubstituted isoquinolines, substituted and unsubstituted anthracenes, substituted and unsubstituted furans, substituted and unsubstituted pyrroles and substituted and unsubstituted thiophenes.

The starting compound must be chosen such that the desired product can be obtained by hydrogenation of one or more unsaturated C—C bonds. For example, it is possible to prepare saturated fatty acids by hydrogenation of mono- or of polyunsaturated fatty acids or from mixture of these two.

It is also possible to prepare substituted saturated compounds from compounds in which at least one substituent is newly formed under the hydrogenation conditions according to the invention. Thus, for example, it is possible to prepare saturated alcohols by hydrogenation of unsaturated aldehydes. In this case, in addition to the hydrogenation according to the invention of the C—C double bond, a carbonyl group is also additionally converted into a hydroxymethyl group. Other examples are the preparation of saturated amines from unsaturated nitriles or nitro compounds.

In a preferred embodiment, cyclohexanes with 0 to 6 substituents from the series consisting of alkyl, cycloalkyl, aryl, alkenyl, alkinyl F, Cl, Br, I, NO₂, NH₂, NHalkyl, NHaryl, Nalkyl₂, Naryl₂, OH, HS, alkylS, arylS, S═C, alkyl-CO—O, aryl-CO—O, alkyl-SO, aryl-SO, alkyl-SO₂, aryl-SO₂, alkyl-SO₃, aryl-SO₃, CN, O═Calkyl, O═Caryl, HOOC, H₂NOC, alkylOOC, arylOOC, alkylO, Sialkyl₃, Sialkyl₂aryl, Sialkylaryl₂, cycloalkylO, arylO can be obtained as products. The substituent can optionally also be substituted by one or more groups from the empires [sic] consisting of alkyl, cycloalkyl, aryl, alkenyl, alkinyl F, Cl, Br, I, NO2, NH2, NHalkyl, NHaryl, Nalkyl₂, Naryl₂, OH, HS, alkyl-S, aryl-S, S═C, alkyl-CO—O, aryl-CO—O, alkyl-SO, aryl-SO, alkyl-SO₂, aryl-SO₂, alkyl-SO₃, aryl-SO₃, CN, O═Calkyl, O═Caryl, HOOC, H₂NOC, alkylOOC, arylOOC, alkylO, Sialkyl₃, cycloalkylO, arylO radicals or heterocyclic radical. Particularly preferred substituents are methyl, ethyl, propyl, butyl, pentyl, hexyl, octyl, decyl, isopropyl, isobutyl, tert-butyl, hydroxyl, methoxy, ethoxy, hydroxymethyl, hydroxyethyl, amino and aminomethyl radicals. The cyclohexanes according to the invention can also be a constituent of a fused ring system. The fused rings can be alicyclic, heterocyclic or aromatic.

In the preferred embodiment, benzene and derivatives thereof can be employed in particular as the starting compound. However, it is also possible to employ other starting compounds, for example cyclohexenes. The substituents can be positioned geminally, vicinally or at a larger distance with respect to one another on the cyclohexane ring. In the preparation of polysubstituted cyclohexanes, it is possible to obtain various products, in particular various stereoisomers, by hydrogenation of a polysubstituted benzene.

In an embodiment which is likewise preferred, saturated heterocyclic compounds with 0 to 6 substituents from the series consisting of alkyl, cycloalkyl, aryl, alkenyl, alkinyl F, Cl, Br, I, NO₂, NH₂, NHalkyl, NHaryl, Nalkyl₂, Naryl₂, OH, HS, alkylS, arylS, S═C, alkyl-CO—O, aryl-CO—O, alkyl-SO, aryl-SO, alkyl-SO₂, aryl-SO₂, alkyl-SO₃, aryl-SO₃, CN, O═Calkyl, O═Caryl, HOOC, H₂NOC, alkylOOC, arylOOC, alkylO, Sialkyl₃, Sialkyl₂aryl, Sialkylaryl₂, cycloalkylO, arylO can be obtained as products by the process according to the invention. The substituent can optionally also be substituted by one or more groups from the series consisting of alkyl, cycloalkyl, aryl, alkenyl, alkinyl F, Cl, Br, I, NO₂, NH₂, NHalkyl, NHaryl, Nalkyl₂, Naryl2, OH, HS, alkyl-S, aryl-S, S═C, alkyl-CO—O, aryl-CO—O, alkyl-SO, aryl-SO, alkyl-SO₂, aryl-SO₂, alkyl-SO₃, aryl-SO₃, CN, O═Calkyl, O═Caryl, HOOC, H₂NOC, alkylOOC, arylOOC, alkylO, Sialkyl₃, cycloalkylO, arylO radicals or heterocyclic radical. Particularly preferred substituents are methyl, ethyl, propyl, butyl, pentyl, hexyl, octyl, decyl, isopropyl, isobutyl, tert-butyl, hydroxyl, methoxy, ethoxy, hydroxymethyl, hydroxyethyl, amino and aminomethyl radicals. It is also possible for the hydrogenated heterocyclic radical to be in a fused ring system. The fused rings can be alicyclic, heterocyclic or aromatic.

These compounds are preferably prepared by hydrogenation from the aromatic compounds on which they are based. However, it is also possible to use other unsaturated compounds as starting substances.

According to the preferred embodiment, for example, tetrahydrofuran and its derivatives, pyrrolidine and its derivatives, tetrahydrothiophene and its derivatives, sulfolane and its derivatives, tetrahydroquinoline and its derivatives, piperazine and its derivatives and piperidine and its derivatives are prepared.

In an embodiment which is likewise preferred, it is possible for saturated organic compounds of the general formula H₃C—(CH₂)ₙ-X to be obtained as products, wherein n is an integer between 1 and 30, preferably between 4 and 25, and X is a functional group from the series consisting of cycloalkyl, aryl, H, F, Cl, Br, I, NO₂, NH₂, NHalkyl, NHaryl, Nalkyl₂, Naryl₂, OH, HS, alkylS, arylS, S═C, alkyl-CO—O, aryl-CO—O, alkyl-SO, aryl-SO, alkyl-SO₂, aryl-SO₂, alkyl-SO₃, aryl-SO₃, CN, O═Calkyl, O═Caryl, HOOC, H₂NOC, alkylOOC, arylOOC, alkylO, Sialkyl₃, Sialkyl₂aryl, Sialkylaryl₂, cycloalkylO, arylO. The preparation of completely saturated fats, fatty acids, fatty alcohols, fatty amines, fatty acid esters, fatty acid nitriles and mixtures of these compounds is particularly preferred.

In an embodiment which is likewise preferred, it is possible to obtain mixtures of completely and/or partly saturated fats, fatty acids and/or fatty acid esters. Such a process is conventionally called hydrogenation of fats in industry. In the hydrogenation of fats, mixtures of unsaturated and saturated fats, unsaturated and saturated fatty acids and/or unsaturated and saturated fatty acid esters, usually in the form of oils or low-melting mixtures, are subjected to catalytic hydrogenation. Since the melting point of the product mixture increases with an increasing degree of saturation, high-melting, low-melting or liquid product mixtures are obtained, depending on the degree of hydrogenation. The fats, fatty acids and fatty acid esters can be unbranched or branched and substituted or unsubstituted. Preferred esters are methyl, ethyl, propyl, butyl, pentyl, hexyl, octyl, decyl and glyceryl esters.

It is also possible to obtain completely or partly saturated polymers as products by the process according to the invention. Polymers which contain C—C multiple bonds in the polymer are used as starting substances.

Example [sic] of compounds which are preferably to be prepared by the process according to the invention are cyclohexane, cyclohexanol, cyclohexylamine, cyclohexyl methyl ether, methylcyclohexane, ethylcyclohexane, isopropylcyclohexane, chlorocyclohexane, dihydroxycyclohexane, methyltetrahydrofuran, chlorotetrahydrofuran, 3-methylpiperidine, 4-methylpiperidine, 3-aminopiperidine, 1-methyl-4-Piperidinol, lauric acid, lauric acid methyl ester, palmitic acid, palmitic acid methyl ester, stearic acid, stearic acid methyl ester, dodecanol, hexadecanol or octadecanol.

Regardless of what type of completely or partly saturated compounds is to be prepared, according to the invention it is possible to prepare only one saturated compound in a reaction. However, it is also possible to prepare mixtures of various saturated compounds by the process according to the invention. These mixtures can be obtained, for example, by non-selective hydrogenation of starting substances which contain several C—C multiple bonds which can be hydrogenated or at least one C—C multiple bond and at least one other group which can be hydrogenated, or by hydrogenation of a mixture of various starting compounds.

Depending on the starting compound, it is possible to carry out the process according to the invention in the liquid phase or in the gas phase. It depends greatly here on what starting compounds are used. The process can be carried out in the liquid phase only if the compound to be hydrogenated is liquid or soluble in a solvent under the reaction conditions.

In many cases it is preferable to carry out the reaction in the presence of a solvent. All the usual solvents can in principle be employed, as long as they do not interfere in the hydrogenation reaction. Examples of conventional solvents are water, dioxane, acetone, methyl ethyl ketone, tetrahydrofuran, cyclohexane, methanol, ethanol, n-propanol, isopropanol, n-butanol, cyclohexanol, ethylene glycol, 1,4-butanediol, 1,6-hexanediol, ethylene glycol methyl ether, ethylene glycol ethyl ether, ethylene glycol dimethyl ether or triethylene glycol methyl ether. Mixtures of different solvents are also possible. The presence of one or more solvents can lead on the one hand to the operating parameters, such as pressure and temperature, lying in more moderate ranges than in the solvent-free procedure, or on the other hand to the reaction being rendered possible in the first place. On the other hand, by skilful choice of the solvents, the selectivity of the hydrogenation reaction can be increased. Preferred solvents are alcohols, in particular methanol and isopropanol, and also toluene, tetrahydrofuran or cyclohexane.

The process according to the invention is preferably carried out under an increased hydrogen pressure. The hydrogen pressures in the hydrogenation are conventionally in a range between 1 and 300 bar, preferably between 2 and 150 bar. In the hydrogenation of aromatic compounds and/or in the continuous procedure, as a rule rather higher pressures must be used than in the hydrogenation of non-aromatic starting substances or if the hydrogenation is carried out in the batch process..

The hydrogenation can be carried out in a temperature range between 0° C. and approximately 300° C., preferably between room temperature and 200° C., in particular between 25 and 150° C., depending on the particular unsaturated starting compound. In the continuous procedure, higher temperatures are usually required than if the corresponding hydrogenation is carried out in the batch process.

By varying the reaction conditions in the preparation of completely or partly saturated organic compounds by catalytic hydrogenation of unsaturated compounds, it is possible also to hydrogenate or not to hydrogenate other groups which can be hydrogenated and are present in the starting compound. It is thus possible on the one hand to obtain as products saturated organic compounds which contain different functional groups to the unsaturated starting compound. On the other hand it is also possible to obtain saturated compounds which contain further groups which can be hydrogenated as substituents.

The preparation of the completely or partly saturated organic compounds can in many cases preferably be carried out continuously in the fixed bed process or semi-continuously. The process can be carried out here in the so-called trickle bed process or in the liquid phase process. The substance or solution to be hydrogenated can be passed accordingly through the catalyst bed from the top or from the bottom. A co-current or a counter-current process can be employed here in a known manner. The catalyst loads can conventionally be in the range between 0.05 and 20 kg of unsaturated starting compound per kg of catalyst and hour.

In the case of the continuous procedure, it is also possible to carry out the hydrogenation in two or more stages. For example, the hydrogenation can be carried out in a first stage at a temperature in the range between 20 and 60° C., and can be completed in a second stage at a temperature in the range from 50 to 100° C. The formation of by-products, for example, can be reduced in this manner.

However, the invention also provides for carrying out the hydrogenation in the suspension process, or in the batch process in the manner in which the catalyst is arranged in a fixed form in a catalyst basket. Suitable reactors for the procedures mentioned are known from the prior art. In this case the amount of hydrogenation catalyst employed is usually non-critical. However, amounts of catalyst which are too low lead to long reaction times, while amounts of catalyst which are too high as a rule are uneconomical. According to the invention, for example, between 0.1 and 40 wt. % of catalyst moist weight, based on the weight of unsaturated starting compound to be hydrogenated, is employed, preferably 0.1-30 wt. %, particularly preferably 0.5-20 wt. %.

The process according to the invention for the preparation of completely or partly saturated organic compounds by catalytic hydrogenation of unsaturated organic compounds with the aid of Raney catalysts in the form of hollow bodies has the following advantages:

The Raney catalyst in the form of hollow bodies used according to the invention has a significantly lower bulk density than the Raney catalysts used hitherto. As a result, considerably less catalyst material is required than in the processes known hitherto.

In spite of the significantly smaller amount of catalyst material, the preparation of saturated organic compounds can be carried out with high conversion rates, very good yields and very good space/time yields.

The catalyst employed in the process according to the invention has a very good strength. This results in a very good hydrogenation activity which lasts a long time, so that long running times without interruptions are achieved in continuous operation.

Because of its state, the catalyst is easy to separate off from the reaction medium after the reaction.

### USE EXAMPLE 1

The catalytic activity [sic] of the catalyst of examples 1 to 7 during hydrogenation of butinediol (BID) to 1,4-butanediol (BDO) and butenediol (BED) were compared. For this purpose, 40 ml catalyst (from 35 to 73 grams of the corresponding catalysts) were introduced into a tube reactor and tested in a trickle phase. The temperature of the reaction was 150° C., the concentration of butinediol in water was 50 wt. %, the pH of the reaction solution was brought to 7 with NaHCO₃ and the pressure of the reaction was from 35 to 60 bar. The throughput of hydrogen was 82.5 l/h and the throughput of butinediol was 0.20 to 1.7 g butinediol/h·ml of catalyst. The product mixture was analysed by GC.

### EXAMPLE 1

A free-flowing, pelletable catalyst mixture was prepared in accordance with the instructions in EP 0 648 534 A1 for a catalyst of 1000 grams of 50% Ni and 50% Al alloy powder (this alloy was melted in an induction furnace and sprayed with water), 75 grams of pure nickel powder (99% Ni and d50=21 μm) and 50 grams of ethylene-bis-stearoylamide. Tablets with a diameter of 3 mm and a thickness of 3 mm were compressed from this mixture. The shaped articles were calcined at 700° C. for 2 hours. After the calcining, the tables were activated for 2 hours at 80° C. in 20% sodium hydroxide solution. 40 ml (66.6 grams) of this catalyst were tested in accordance with use example 1 and the results of this experiment are shown in table 1.

TABLE 1
The results of example 1

| | | | | Sel. | Activity | Activity |
|---|---|---|---|---|---|---|
| Pressure | Throughput | | Sel. | Without | per | per |
| [bar] | [g BID/ (h · ml cat.)] | Conversion [%] | [%]^{A} | BED [%]^{B} | weight^{C} | volume^{D} |
| 35 | 0.20 | 99.4 | 75.7 | 85.5 | 2.389 | 3.977 |
| 60 | 0.20 | 99.7 | 63.1 | 70.3 | 2.994 | 4.984 |

^{A}% selectivity
^{B}% selectivity, where the amount of BED is not counted
^{C}Activity per weight = [g BDO/h · g cat]
^{D}Activity per volume = [g BDO/h · ml cat]

### EXAMPLE 2

A free-flowing, pelletable catalyst mixture was prepared in accordance with the instructions in EP 0 648 534 A1 for a catalyst of 1000 grams of 50% Ni and 50% Al alloy powder (this alloy was melted in an induction furnace and sprayed with water), 75 grams of pure nickel powder (99% Ni and d50=21 μm) and 50 grams of ethylene-bis-stearoylamide. Tablets with a diameter of 3 mm and a thickness of 3 mm were compressed from this mixture. The shaped articles were calcined at 700° C. for 2 hours. After the calcining, the tables were activated for 2 hours at 80° C. in 20% sodium hydroxide solution. This catalyst was doped with a sodium molybdate solution and the Mo content of the catalyst at the end was 0.2%. 40 ml (72.8 grams) of this catalyst were tested in accordance with use example 1 and the results of this experiment are shown in table 2.

TABLE 2
The results of example 2

| | | | | Sel. | Activity | Activity |
|---|---|---|---|---|---|---|
| Pressure | Throughput | | Sel. | Without | per | per |
| [bar] | [g BID/ (h · ml cat.)] | Conversion [%] | [%]^{A} | BED [%]^{B} | weight^{C} | volume^{D} |
| 35 | 0.40 | 97.4 | 55.6 | 73.5 | 0.37 | 0.67 |

^{A}% selectivity
^{B}% selectivity, where the amount of BED is not counted
^{C}Activity per weight = [g BDO/h · g cat]
^{D}Activity per volume = [g BDO/h · ml cat]

### EXAMPLE 3

A coating solution was prepared by suspending 1730 grams of 53% Ni and 47% Al alloy powder and 130 grams of pure nickel powder (99% Ni and d50=21 μm) in 1557 ml of an aqueous solution with a content of approx. 2 wt. % polyvinyl alcohol. This suspension was then sprayed on to 1,000 ml of poylstyrene [sic] beads with a diameter of about approx. 2 mm, while these were suspended in a stream of air directed upwards. 1 liter of these beads was coated further with an alloy solution. The solution for the second layer comprised 1203 grams of 53% Ni and 47% Al alloy powder, 90 grams of pure nickel powder (99% Ni and d50=21 μm) and 1083 ml of an aqueous solution with a content of approx. 2 wt. % polyvinyl alcohol. This suspension was then sprayed on to 1,000 ml of the abovementioned polystyrene beads precoated with Ni/Al, while these were suspended in a stream of air (nitrogen and other gases can also be used) directed upwards. After the polystyrene beads had been coated with the abovementioned solutions, the beads were heated to 500° C. in order to burn out the polystyrene. The Ni/Al hollow spheres were then heated to 800° C. in order to sinter together the alloy particles and nickel powder. The hollow spheres were then activated in a 20 wt. % sodium hydroxide solution for approx. 1.5 h at 80° C. The activated hollow spheres obtained had a diameter of about approx. 3.3 mm and a shell thickness of about approx. 700 μm. This catalyst was doped with a sodium molybdate solution and the Mo content of the catalyst at the end was 0.3%. 40 ml (36.07 grams) of this catalyst were tested in accordance with use example 1 and the results of this experiment are shown in table 3.

TABLE 3
The results of example 3

| | | | | Sel. | Activity | Activity |
|---|---|---|---|---|---|---|
| Pressure | Throughput | | Sel. | Without | per | per |
| [bar] | [g BID/ (h · ml cat.)] | Conversion [%] | [%]^{A} | BED [%]^{B} | weight^{C} | volume^{D} |
| 60 | 0.80 | 99.8 | 83.0 | 89.5 | 0.89 | 0.798 |

^{A}% selectivity
^{B}% selectivity, where the amount of BED is not counted
^{C}Activity per weight = [g BDO/h · g cat]
^{D}Activity per volume = [g BDO/h · ml cat]

### EXAMPLE 4

A coating solution was prepared by suspending 1730 grams of 48.5% Ni, 50.1% Al, 0.9% Cr and 0.5% Fe alloy powder alloy powder and 130 grams of pure nickel powder (99% Ni and d50=21 μm) in 1557 ml of an aqueous solution with a content of approx. 2 wt. % polyvinyl alcohol. This suspension was then sprayed on to 1,000 ml of poylstyrene [sic] beads with a diameter of about approx. 2 mm, while these were suspended in a stream of air directed upwards. 1 liter of these beads was coated further with an alloy solution. The solution for the second layer comprised 1203 grams of 48.5% Ni, 50.5% Al, 0.9% Cr and 0.5% Fe alloy powder, 90 grams of pure nickel powder (99% Ni and d50=21 μm) and 1083 ml of an aqueous solution with a content of approx.

2 wt. % polyvinyl alcohol. This suspension was then sprayed on to 1,000 ml of the abovementioned polystyrene beads precoated with Ni/Al/Cr/Fe, while these were suspended in a stream of air (nitrogen and other gases can also be used) directed upwards. After the polystyrene beads had been coated with the abovementioned solutions, the beads were heated to 500° C. in order to burn out the polystyrene. The Ni/Al/Cr/Fe hollow spheres were then heated to 800° C. in order to sinter together the alloy particles and nickel powder. The hollow spheres were then activated in a 20 wt. % sodium hydroxide solution for approx. 1.5 h at 80° C. The activated hollow spheres obtained had a diameter of about approx. 3.3 mm and a shell thickness of about approx. 700 μm. 40 ml (32.88 grams) of this catalyst were tested in accordance with use example 1 and the results of this experiment are shown in table 4.

TABLE 4
The results of example 4

| | | | | Sel. | Activity | Activity |
|---|---|---|---|---|---|---|
| Pressure | Throughput | | Sel. | Without | per | per |
| [bar] | [g BID/ (h · ml cat.)] | Conversion [%] | [%]^{A} | BED [%]^{B} | weight^{C} | volume^{D} |
| 60 | 0.80 | 96.12 | 63.14 | 79.23 | 0.94 | 0.774 |
| 60 | 0.80 | 94.13 | 63.00 | 82.93 | 0.92 | 0.754 |
| 60 | 1.60 | 71.63 | 48.10 | 74.17 | 1.40 | 1.149 |
| 60 | 1.60 | 78.28 | 49.53 | 73.66 | 1.53 | 1.256 |

^{A}% selectivity
^{B}% selectivity, where the amount of BED is not counted
^{C}Activity per weight = [g BDO/h · g cat]
^{D}Activity per volume = [g BDO/h · ml cat]

### EXAMPLE 5

A free-flowing, pelletable catalyst mixture was prepared in accordance with the instructions in EP 0 648 534 A1 for a catalyst of 1000 grams of 50% Ni and 50% Al alloy powder (this alloy was melted in an induction furnace and sprayed with water), 75 grams of pure nickel powder (99% Ni and d50=21 μm) and 50 grams of ethylene-bis-stearoylamide. Tablets with a diameter of 3 mm and a thickness of 3 mm were compressed from this mixture. The shaped articles were calcined at 700° C. for 2 hours. After the calcining, the tables were activated for 2 hours at 80° C. in 20% sodium hydroxide solution. 40 ml (70.3 grams) of this catalyst were tested in accordance with use example 1 and the results of this experiment are shown in table 5.

TABLE 5
The results of example 5

| | | | | Sel. | Activity | Activity |
|---|---|---|---|---|---|---|
| Pressure | Throughput | | Sel. | Without | per | per |
| [bar] | [g BID/ (h · ml cat.)] | Conversion [%] | [%]^{A} | BED [%]^{B} | weight^{C} | volume^{D} |
| 60 | 0.80 | 83.6 | 36.6 | 66.3 | 0.38 | 0.669 |
| 60 | 0.80 | 88.2 | 36.8 | 65.7 | 0.40 | 0.706 |
| 60 | 1.60 | 62.8 | 28.6 | 56.3 | 0.57 | 1.005 |

^{A}% selectivity
^{B}% selectivity, where the amount of BED is not counted
^{C}Activity per weight = [g BDO/h · g cat]
^{D}Activity per volume = [g BDO/h · ml cat]

### EXAMPLE 6

A free-flowing, pelletable catalyst mixture was prepared in accordance with the instructions in EP 0 648 534 A1 for a catalyst of 1000 grams of 50% Ni and 50% Al alloy powder (this alloy was melted in an induction furnace and sprayed with water), 75 grams of pure nickel powder (99% Ni and d50=21 μm) and 50 grams of ethylene-bis-stearoylamide. Tablets with a diameter of 3 mm and a thickness of 3 mm were compressed from this mixture. The shaped articles were calcined at 700° C. for 2 hours. After the calcining, the tables were activated for 2 hours at 80° C. in 20% sodium hydroxide solution. This catalyst was doped with a sodium molybdate solution and the Mo content of the catalyst at the end was 0.2%. 40 ml (70.9 grams) of this catalyst were tested in accordance with use example 1 and the results of this experiment are shown in table 6.

TABLE 6
The results of example 6

| | | | | Sel. | Activity | Activity |
|---|---|---|---|---|---|---|
| Pressure | Throughput | | Sel. | Without | per | per |
| [bar] | [g BID/ (h · ml cat.)] | Conversion [%] | [%]^{A} | BED [%]^{B} | weight^{C} | volume^{D} |
| 60 | 0.80 | 95.3 | 51.4 | 75.1 | 0.43 | 0.763 |
| 60 | 0.80 | 92.1 | 47.5 | 73.1 | 0.42 | 0.737 |
| 60 | 1.60 | 71.4 | 28.9 | 48.5 | 0.64 | 1.142 |

^{A}% selectivity
^{B}% selectivity, where the amount of BED is not counted
^{C}Activity per weight = [g BDO/h · g cat]
^{D}Activity per volume = [g BDO/h · ml cat]

### EXAMPLE 7

A coating solution was prepared by suspending 1730 grams of 53% Ni and 47% Al alloy powder and 130 grams of pure nickel powder (99% Ni and d50=21 μm) in 1557 ml of an aqueous solution with a content of approx. 2 wt. % polyvinyl alcohol. This suspension was then sprayed on to 1,000 ml of polystyrene beads with a diameter of about approx. 2 mm, while these were suspended in a stream of air directed upwards. 1 liter of these beads was coated further with an alloy solution. The solution for the second layer comprised 1203 grams of 53% Ni and 47% Al alloy powder, 90 grams of pure nickel powder (99% Ni and d50=21 μm) and 1083 ml of an aqueous solution with a content of approx. 2 wt. % polyvinyl alcohol. This suspension was then sprayed on to 1,000 ml of the abovementioned polystyrene beads precoated with Ni/Al, while these were suspended in a stream of air (nitrogen and other gases can also be used) directed upwards. After the polystyrene beads had been coated with the abovementioned solutions, the beads were heated to 500° C. in order to burn out the polystyrene. The Ni/Al hollow spheres were then heated to 800° C. in order to sinter together the alloy particles and nickel powder. The hollow spheres were then activated in a 20 wt. % sodium hydroxide solution for approx. 1.5 h at 80° C. The activated hollow spheres obtained had a diameter of about approx. 3.3 mm and a shell thickness of about approx. 700 μm. 40 ml (34.62 grams) of this catalyst were tested in accordance with use example 1 and the results of this experiment are shown in table 7.

TABLE 7
The results of example 7

| | | | | Sel. | Activity | Activity |
|---|---|---|---|---|---|---|
| Pressure | Throughput | | Sel. | Without | per | per |
| [bar] | [g BID/ (h · ml cat.)] | Conversion [%] | [%]^{A} | BED [%]^{B} | weight^{C} | volume^{D} |
| 60 | 0.80 | 99.5 | 71.0 | 81.0 | 0.920 | 0.796 |
| 60 | 1.60 | 91.3 | 44.7 | 67.9 | 1.688 | 1.461 |

^{A}% selectivity
^{B}% selectivity, where the amount of BED is not counted
^{C}Activity per weight = [g BDO/h · g cat]
^{D}Activity per volume = [g BDO/h · ml cat]

### USE EXAMPLE 2

The catalytic activities of the catalyst of example 8 during hydrogenation of butinediol (BID) to 1,4-butanediol (BDO) and butenediol (BED) were investigated. For this purpose, 40 ml of catalyst were introduced into a tube reactor and tested in a bubble column process. The temperature of the reaction was 150° C., the concentration of butinediol in water was 50 wt. %, the pH of the reaction solution was brought to 7 with NaHCO₃ and the pressure of the reaction was 60 bar. The throughput of hydrogen was 82.5 l/h and the throughput of butinediol was 0.40 g butinediol/h·ml of catalyst. The product mixture was analysed by GC.

### EXAMPLE 8

A free-flowing, pelletable catalyst mixture was prepared in accordance with the instructions in EP 0 648 534 A1 for a catalyst of 1000 grams of 50% Ni and 50% Al alloy powder (this alloy was melted in an induction furnace and sprayed with water), 75 grams of pure nickel powder (99% Ni and d50=21 μm) and 50 grams of ethylene-bis-stearoylamide. Tablets with a diameter of 3 mm and a thickness of 3 mm were compressed from this mixture. The shaped articles were calcined at 700° C. for 2 hours. After the calcining, the tables were activated for 2 hours at 80° C. in 20% sodium hydroxide solution. This catalyst was doped with a sodium molybdate solution and the Mo content of the catalyst at the end was 0.2%. 40 ml (72.8 grams) of this catalyst were tested in accordance with use example 2 and the results of this experiment are shown in table 8.

TABLE 8
The results of example 8

| | | | | Sel. | Activity | Activity |
|---|---|---|---|---|---|---|
| Pressure | Throughput | | Sel. | Without | per | per |
| [bar] | [g BID/ (h · ml cat.)] | Conversion [%] | [%]^{A} | BED [%]^{B} | weight^{C} | volume^{D} |
| 60 | 0,40 | 99,4 | 75,7 | 85,5 | 2,18 | 3,97 |
| 60 | 0,40 | 99,7 | 63,1 | 70,3 | 2,74 | 4,99 |

^{A}% selectivity
^{B}% selectivity, where the amount of BED is not counted
^{C}Activity per weight = [g BDO/h · g cat]
^{D}Activity per volume = [g BDO/h · ml cat]

### USE EXAMPLE 3

The catalytic activity [sic] of the catalyst of examples 9 and 10 during hydrogenation of a mixture of aromatics from benzene were compared. For this purpose, 11.6 to 11.0 grams of catalyst were introduced into a flask in an autoclave and tested in a liquid phase. The temperature of the reaction was 180° C., the amount of aromatics was 200 grams, The solution was stirred at 1000 rpm and the pressure of the reaction was 35 bar. The activity was calculated by H₂ uptake.

### EXAMPLE 9

11.6 grams of a commercial 10%NI/Al₂O₃ catalyst were tested in accordance with use example 3. This catalyst showed an activity per catalyst volume of 15.81 ml H₂/ml cat·h and an activity per catalyst weight of 18.82 ml H₂/g cat·h.

### EXAMPLE 10

A coating solution was prepared by suspending 1730 grams of 53% Ni and 47% Al alloy powder and 130 grams of pure nickel powder (99%.Ni and d50=21 μm) in 1557 ml of an aqueous solution with a content of approx. 2 wt. % polyvinyl alcohol. This suspension was then sprayed on to 1,000 ml of polystyrene beads with a diameter of about approx. 2 mm, while these were suspended in a stream of air directed upwards. 1 liter of these beads was coated further with an alloy solution. The solution for the second layer comprised 1203 grams of 53% Ni and 47% Al alloy powder, 90 grams of pure nickel powder (99% Ni and d50=21 μm) and 1083 ml of an aqueous solution with a content of approx. 2 wt. % polyvinyl alcohol. This suspension was then sprayed on to 1,000 ml of the abovementioned polystyrene beads precoated with Ni/Al, while these were suspended in a stream of air (nitrogen and other gases can also be used) directed upwards. After the polystyrene beads had been coated with the abovementioned solutions, the beads were heated to 500° C. in order to burn out the polystyrene. The Ni/Al hollow spheres were then heated to 800° C. in order to sinter together the alloy particles and nickel powder. The hollow spheres were then activated in a 20 wt. % sodium hydroxide solution for approx. 1.5 h at 80° C. The activated hollow spheres obtained had a diameter of about approx. 3.3 mm and a shell thickness of about approx. 700 μm. 11.0 grams of this catalyst were tested in accordance with use example 3. This catalyst showed an activity per catalyst volume of 29.16 ml H₂/ml cat·h and an activity per catalyst weight of 36.45 ml H₂/g cat h.

1. Process for the preparation of completely or partly saturated organic compounds by catalytic hydrogenation of unsaturated organic compounds with hydrogen or hydrogen-containing gas mixtures in the presence of a shaped Raney catalyst as the hydrogenation catalyst, characterized in that the Raney catalyst is in the form of hollow bodies.

2. Process according to claim 1, characterized in that the Raney catalysts in the form of hollow bodies comprise nickel, cobalt, copper, iron, platinum, palladium, ruthenium or mixtures of these metals as catalytically active constituents.

3. Process according to claim 1 or 2, characterized in that the Raney catalyst is in the form of hollow spheres.

4. Process according to claim 1 or 3, characterized in that the bulk density of the Raney catalysts used is in the range from 0.3 g/ml to 1.3 g/ml.

5. Process according to claim 1 or 4, characterized in that the catalyst shaped articles used have a diameter in the range from 0.05 to 20 mm.

6. Process according to one or more of claims 1 to 6, characterized in that the catalyst shaped articles used have a shell thickness in the range from 0.05 to 7 mm, preferably 0.1 mm to 5 mm.

7. Process according to one or more of claims 1 to 6, characterized in that the activated catalyst shaped articles used in the process comprise an inorganic binder.

8. Process according to one or more of claims 1 to 6, characterized in that the activated catalyst shaped articles used in the process comprise no binder.

9. Process according to one or more of claims 1 to 8, characterized in that the Raney catalyst in the form of hollow bodies used is doped with one or more elements from groups 3B to 7B, 8 and 1B of the periodic table, in particular chromium, manganese, iron, vanadium, tantalum, titanium, tungsten, molybdenum, rhenium and/or metals of the platinum group.

10.Process according to one or more of claims 1 to 9, characterized in that the Raney catalyst in the form of hollow bodies used is doped with one or more elements from groups 1A, 2A, 2B and/or 3A of the periodic table and/or germanium, tin, lead, antimony or bismuth.

11. Process according to one or more of claims 1 to 10, characterized in that the hydrogenation is carried out in a fixed bed or suspension reactor in continuous operation.

12. Process according to one or more of claims 1 to 10, characterized in that the hydrogenation is carried out in the batch process.

13.Process according to one or more of claims 1 to 12, characterized in that cyclohexanes with 0 to 6 substituents from the series consisting of alkyl, cycloalkyl, aryl, alkenyl, alkinyl F, Cl, Br, I, NO₂, NH₂, NHalkyl, NHaryl, Nalkyl₂, Naryl₂, OH, HS, alkylS, arylS, S═C, alkyl-CO—O, aryl-CO—O, alkyl-SO, aryl-SO, alkyl-SO₂, aryl-SO₂, alkyl-SO₃, aryl-SO₃, CN, O═Calkyl, O═Caryl, HOOC, H₂NOC, alkylOOC, arylOOC, alkylO, Sialkyl₃, Sialkyl₂aryl, Sialkylaryl₂, cycloalkylO, arylO are obtained as products.

14. Process according to one or more of claims 1 to 12, characterized in that saturated heterocyclic compounds with 0 to 6 substituents from the series consisting of alkyl, cycloalkyl, aryl, alkenyl, alkinyl F, Cl, Br, I, NO₂, NH₂, NHalkyl, NHaryl, Nalkyl₂, Naryl₂, OH, HS, alkylS, arylS, S═C, alkyl-CO—O, aryl-CO—O, alkyl-SO, aryl-SO, alkyl-SO₂, aryl-SO₂, alkyl-SO₃, aryl-SO₃, CN, O═Calkyl, O═Caryl, HOOC, H₂NOC, alkylOOC, arylOOC, alkylO, Sialkyl₃, Sialkyl₂aryl, Sialkylaryl₂, cycloalkylO, arylO are obtained as products.

15. Process according to one or more of claims 1 to 12, characterized in that saturated organic compounds of the general formula H₃C—(CH₂)ₙ-X are obtained as products, wherein n is an integer between 1 and 30, preferably between 4 and 25, and X is a functional group from the series consisting of cycloalkyl, aryl, F, Cl, Br, I, NO₂, NH₂, NHalkyl, NHaryl, Nalkyl₂, Naryl₂, OH, HS, alkylS, arylS, S═C, alkyl-CO—O, aryl-CO—O, alkyl-SO, aryl-SO, alkyl-SO₂, aryl-SO₂, alkyl-SO₃, aryl-SO₃, CN, O═Calkyl, O═Caryl, HOOC, H₂NOC, alkylOOC, arylOOC, alkylO, Sialkyl₃, Sialkyl₂aryl, Sialkylaryl₂, cycloalkylO, arylO.

16. Process according to one or more of claims 1 to 12, characterized in that mixtures of completely and/or partly saturated fats, fatty acids, fatty nitriles, fatty amines and/or fatty acid esters are obtained.

17. Process according to one or more of claims 1 to 12, characterized in that the products are butanediol or/and butenediol from the hydrogenation of butinediol.

18. Process according to one or more of claims 1 to 12, characterized in that the products are butanediol or/and butenediol from the hydrogenation of butinediol in the presence of the shell-activated tablets of the Raney type alloy.

19. Process according to one or more of claims 1 to 12, characterized in that the products are butanediol or/and butenediol from the hydrogenation of butinediol in the presence of the shell-activated tablets of the Raney type alloy which is doped doped [sic] with one or more elements from groups 3B to 7B, 8 and 1B of the periodic table, in particular chromium, manganese, iron, vanadium, tantalum, titanium, tungsten, molybdenum, rhenium and/or metals of the platinum group.

20. Process according to one or more of claims 1 to 12, characterized in that the products are butanediol or/and butenediol from the hydrogenation of butinediol in the presence of the shell-activated tablets of the Raney type alloy which is doped with one or more elements from groups 1A, 2A, 2B and/or 3A of the periodic table and/or germanium, tin, lead, antimony or bismuth.

21. Process according to one or more of claims 1 to 12, characterized in that the products are saturated rings from the hydrogenation of aromatics.

22. Process according to one or more of claims 1 to 12, characterized in that the products are saturated rings from the hydrogenation of aromatics in the presence of the shell-activated tablets of the Raney type alloy.

23. Process according to one or more of claims 1 to 12, characterized in that the products are saturated rings from the hydrogenation of aromatics in the presence of the shell-activated tablets of the Raney type alloy which is doped with one or more elements from groups 3B to 7B, 8 and 1B of the periodic table, in particular chromium, manganese, iron, vanadium, tantalum, titanium, tungsten, molybdenum, rhenium and/or metals of the platinum group.

24. Process according to one or more of claims 1 to 12, characterized in that the products are saturated rings from the hydrogenation of aromatics in the presence of the shell-activated tablets of the Raney type alloy which is doped with one or more elements from groups 1A, 2A, 2B and/or 3A of the periodic table and/or germanium, tin, lead, antimony or bismuth.
